(19) United States
(12) Patent Application Publication
(10) Pub. No.: US 2013/0197333 A1
(43) Pub. Date: Aug. 1, 2013

(54) ANALYTE SENSOR

(71) Applicant: James R. Petisce, Westford, MA (US)

(72) Inventor: James R. Petisce, Westford, MA (US)

(73) Assignee: EDWARDS LIFESCIENCES CORPORATION, IRVINE, CA (US)

(21) Appl. No.: 13/641,762

(22) PCT Filed: Jun. 28, 2011
(86) PCT No.: PCT/US11/42151

§ 371 Date: Feb. 28, 2013

Related U.S. Application Data
(60) Provisional application No. 61/360,215, filed on Jun. 30, 2010.

(51) Int. Cl. C12Q 1/00; A61B 5/1486; A61B 5/00; A61B 5/145

(52) U.S. Cl. CPC C12Q 1/006; A61B 5/14532; A61B 5/14865; A61B 5/14539; A61B 5/14535; A61B 5/4839
USPC 600/347; 204/403.1; 205/778; 204/403.11

(57) ABSTRACT

The present disclosure relates generally to an electrochemical sensor comprising a membrane layer comprising one or both of an active enzymatic portion and an inactive-enzymatic or non-enzymatic portion, at least one electrode disposed beneath the membrane and either at least one pH sensor or a hematocrit sensor. The present disclosure also relates to methods of adjusting analyte concentration values using a correction factor based on measured pH values and/or measured hematocrit levels.

### FIELD OF THE INVENTION

The present disclosure relates generally to an electrochemical sensor comprising a membrane layer comprising one or both of an active enzymatic portion and an inactive-enzymatic or non-enzymatic portion, and at least two electrodes disposed beneath the membrane and either at least one pH sensor or a hematocrit sensor. The present disclosure also relates to methods of adjusting analyte concentration values using a correction factor based on measured pH values and/or a measured hematocrit level.

### BACKGROUND

There are a number of known sensors that use an electrochemical cell to provide output signals by which the presence or absence of an analyte in a sample, such as blood, can be determined. For example, in an electrochemical cell, an analyte (or analyte derivative) that is electro-active generates a detectable signal at an electrode, and this signal can be used to detect or measure the presence and/or amount within a biological sample.

In some sensors, an enzyme is provided that reacts with an analyte to be measured, and the byproduct of the reaction is qualified or quantified at the electrode. In one amperometric glucose oxidase-based glucose sensor, immobilized glucose oxidase catalyses the oxidation of glucose to form hydrogen peroxide, which is then quantified by amperometric measurement (for example, change in electrical current) through a polarized electrode.

### SUMMARY

Disclosed and described herein are analyte sensors and sensor assemblies comprising either at least one pH sensor or a hematocrit sensor positioned in proximity to electrodes and methods for providing a correction factor for adjusting a glucose concentration value based on a measured pH value and/or a measured hematocrit level.

In a first embodiment, an analyte sensor is provided. The analyte sensor includes a membrane comprising one or both of an active enzymatic portion and an inactive-enzymatic or non-enzymatic portion, at least two electrodes disposed beneath the membrane, and at least one pH sensor positioned in proximity to the at least one electrode.

In one aspect of the first embodiment, the at least one pH sensor is disposed beneath the membrane.

In a second aspect, alone or in combination with the previous aspect of the first embodiment, the at least two electrodes comprise a working electrode and a blank electrode, and the membrane is partitioned over the working electrode and the blank electrode.

In a third aspect, alone or in combination with the previous aspect of the first embodiment, the working electrode is disposed under the active enzymatic portion of the membrane and the blank electrode is disposed under the inactive-enzymatic or non-enzymatic portion of the membrane.

In a fourth aspect, alone or in combination with any one of the second or third aspects of the first embodiment, the membrane is partitioned over the working electrode associated with the active enzymatic portion and the blank electrode associated with the inactive-enzymatic or non-enzymatic portion.

In a fifth aspect, alone or in combination with the third aspect of the first embodiment, the at least one pH sensor is: (i) positioned in closer proximity to the working electrode than the blank electrode; (ii) positioned in closer proximity to the blank electrode than the working electrode; or (iii) positioned at an equal distance from the working electrode and the blank electrode.

In a sixth aspect, alone or in combination with any one of the previous aspects of the first embodiment, the active enzymatic portion of the membrane comprises glucose oxidase.

In a seventh aspect, alone or in combination with any one of the previous aspects of the first embodiment, the at least one electrode and the pH sensor is disposed on a first surface of a sensor substrate.

In an eighth aspect, alone or in combination with any one of the previous aspects of the first embodiment, the at least one electrode is disposed on a first surface of a sensor substrate and the pH sensor is disposed on a second surface of a sensor substrate.

In a ninth aspect, alone or in combination with any one of the previous aspects of the first embodiment, the membrane further comprises at least one of an electrode layer, an interferent layer, and a flux limiting layer.

In a tenth aspect of the first embodiment, alone or in combination with any one of the previous aspects of the first embodiment, the at least one pH sensor is configured to determine a pH value of an environment in proximity to one or both of the least two electrodes.

In a second embodiment, a method is provided. The method includes providing an analyte sensor adaptable to an infusion source, the sensor comprising a membrane comprising one or both of an active enzymatic portion and an inactive-enzymatic or non-enzymatic portion, at least one working electrode disposed beneath one or both of the active enzymatic portion and an inactive-enzymatic or non-enzymatic portion, and at least one pH sensor positioned in proximity to one or both of the at least one working electrode. A first signal generated by the at least one electrode for determining a concentration of analyte when in contact with an intravenous sample is obtained, providing an analyte concentration value based on the first signal. A second signal generated by the pH sensor corresponding to a pH value when in contact with bodily fluids is obtained, providing a correction factor based on the second signal, The analyte concentration value is adjusted using the correction factor.

In a first aspect of the second embodiment, the analyte sensor is an intravenous blood glucose sensor (IVBG).

In a second aspect, alone or in combination with any one of the previous aspects of the second embodiment, the correction factor is determined using an algorithm.

In a third aspect, alone or in combination with any one of the previous aspects of the second embodiment, the algorithm comprises a pH correction curve.

In a fourth aspect, alone or in combination with any one of the previous aspects of the second embodiment, the second signal corresponds to one or more of the pH of the infusion source introduced to the analyte sensor or the pH of the intravenous sample.

In a fifth aspect, alone or in combination with any one of the previous aspects of the second embodiment, the pH of the infusion source differs from the pH of the intravenous sample.

In a sixth aspect, alone or in combination with any one of the previous aspects of the second embodiment, the method further comprising obtaining a signal corresponding to a hematocrit level present in the bodily fluid and adjusting the calculated analyte concentration value based on the determined hematocrit level.

In a seventh aspect, alone or in combination with any one of the previous aspects of the second embodiment, further comprises measuring an impedance value of the bodily fluid corresponding to a hematocrit level, calculating a second correction factor based on the measured impedance value, and adjusting the calculated analyte concentration value based on the calculated second correction factor.

In an eighth aspect, alone or in combination with any one of the previous aspects of the second embodiment, the calculated analyte concentration value is adjusted based on the calculated first correction factor and the calculated second correction factor.

In a ninth aspect, alone or in combination with any one of the previous aspects of the second embodiment, the at least one pH sensor is disposed beneath the membrane.

In a tenth aspect, alone or in combination with any one of the previous aspects of the second embodiment, the at least one pH sensor is disposed beneath an ion-sensitive membrane.

In a third embodiment, a system is provided. The system comprises an intravenous analyte sensor adapted for fluid communication with an infusion fluid source and intravenous fluids. The analyte sensor comprises at least one enzyme electrode configured to generate a first signal, corresponding to an analyte concentration value of the intravenous fluid, and at least one pH sensor in proximity to the at least one enzyme electrode, the pH sensor configured to generate a second signal corresponding to a pH value of one or more of the infusion fluid source and the intravenous fluid. The system is configured to adjust the analyte concentration value based on the pH value corresponding to the second signal.

In a fourth embodiment, an analyte sensor is provide. The sensor comprises a membrane comprising an active enzymatic portion and an inactive-enzymatic or non-enzymatic portion; at least two electrodes disposed beneath the membrane; and a hematocrit sensor positioned in proximity to the at least two electrodes.

In a first aspect of the fourth embodiment, the hematocrit sensor is disposed beneath the membrane.

In a second aspect, alone or in combination with any one of the previous embodiments of the fourth embodiment, the at least two electrodes comprises a working electrode and a blank electrode, and the membrane is partitioned over the working electrode and the blank electrode.

In a third aspect, alone or in combination with any one of the previous embodiments of the fourth embodiment, the working electrode is disposed under the active enzymatic portion of the membrane and the blank electrode is disposed under the inactive-enzymatic or non-enzymatic portion of the membrane.

In a fourth aspect, alone or in combination with any one of the previous embodiments of the fourth embodiment, the membrane is partitioned over the working electrode associated with the active enzymatic portion and the blank electrode associated with the inactive-enzymatic or non-enzymatic portion.

In a fifth aspect, alone or in combination with any one of the previous embodiments of the fourth embodiment, the active enzymatic portion of the membrane comprises glucose oxidase.

In a sixth aspect, alone or in combination with any one of the previous embodiments of the fourth embodiment, the working electrode and the hematocrit sensor is disposed on a first surface of a sensor substrate.

In a seventh aspect, alone or in combination with any one of the previous embodiments of the fourth embodiment, the working electrode is disposed on a first surface of a sensor substrate, and the hematocrit sensor is disposed on a second surface of the sensor substrate.

In an eighth aspect, alone or in combination with any one of the previous embodiments of the fourth embodiment, the sensor further comprises at least one pH sensor.

In a fifth embodiment, an analyte sensor is provided. The sensor comprises a substrate having a first surface and a second surface; at least one electrode disposed on the first surface and a hematocrit sensor disposed on the second surface, wherein the at least one electrode is disposed beneath a membrane, the membrane comprising an active enzymatic portion and an inactive-enzymatic or non-enzymatic portion.

In a first aspect of the fifth embodiment, the hematocrit sensor comprises at least two electrodes.

In a second aspect, alone or in combination with any one of the previous embodiments of the fifth embodiment, the at least one electrode comprises a working electrode and a blank electrode, and the membrane is partitioned over the working electrode and the blank electrode.

In a third aspect, alone or in combination with any one of the previous embodiments of the fifth embodiment, the membrane is partitioned over the working electrode associated with the active enzymatic portion and the blank electrode associated with the inactive-enzymatic or non-enzymatic portion.

In a sixth embodiment, an analyte sensor is provided. The sensor comprises a membrane comprising an active enzymatic portion and an inactive-enzymatic or non-enzymatic portion; at least one electrode disposed beneath the membrane; and a hematocrit sensor comprising at least one optical fiber positioned in proximity to the at least two electrodes disposed beneath the membrane.

In a seventh embodiment, a method is provided. The method comprises providing an analyte sensor comprising: a membrane layer comprising one or both of an active enzymatic portion and an inactive-enzymatic or non-enzymatic portion; at least one working electrode disposed beneath one or both of the active enzymatic portion of the membrane and the inactive-enzymatic or non-enzymatic portion of the membrane; and a hematocrit sensor positioned in proximity to one or both of the at least one working electrode; obtaining a first signal generated by the at least one electrode for determining a concentration of an analyte when in contact with an intravenous sample and providing an analyte concentration value based on the first signal; obtaining a second signal generated by the hematocrit sensor corresponding to a hematocrit level of the intravenous sample; providing a correction factor based on the second signal; and adjusting the analyte concentration value using the correction factor.

In a first aspect of the seventh embodiment, the analyte sensor is an intravenous blood glucose sensor (IVBG).

In a second aspect, alone or in combination with any one of the previous embodiments of the seventh embodiment, the correction factor is determined using an algorithm.

In a third aspect, alone or in combination with any one of the previous embodiments of the seventh embodiment, the hematocrit sensor comprises at least two electrodes.

In a fourth aspect, alone or in combination with any one of the previous embodiments of the seventh embodiment, the method further comprises measuring an impedance value of the intravenous sample corresponding to a hematocrit level.

In a fifth aspect, alone or in combination with any one of the previous embodiments of the seventh embodiment, the hematocrit sensor comprises at least one optical fiber.

In a sixth aspect, alone or in combination with any one of the previous embodiments of the seventh embodiment, the method further comprises passing light through the intravenous sample and measuring the transmittance of light through the intravenous sample.

In a seventh aspect, alone or in combination with any one of the previous embodiments of the seventh embodiment, the hematocrit sensor is disposed beneath the membrane.

In an eighth aspect, alone or in combination with any one of the previous embodiments of the seventh embodiment, the hematocrit sensor comprises at least four electrodes.

In a ninth aspect, alone or in combination with any one of the previous embodiments of the seventh embodiment, the hematocrit sensor is disposed on a first surface of a substrate and the working electrode is disposed on a second surface of a substrate.

In a tenth aspect, alone or in combination with any one of the previous embodiments of the seventh embodiment, the hematocrit sensor comprises at least two optical fibers.

### BRIEF DESCRIPTION OF THE DRAWINGS

FIG. 1 is a schematic diagram of a four-electrode biosensor according to an embodiment of the invention.

FIG. 2 is a block diagram of a monitoring system for monitoring the output of an electro-chemical sensor according to one embodiment of the present invention.

FIG. 3A shows a sensor configured with at least one electrode and a pH sensor, according to an embodiment disclosed and described herein.

FIG. 3B is a cross-sectional side view of a sensor configured with a pH sensor in the vicinity of a working electrode, of an embodiment disclosed herein.

FIG. 3C is a cross-sectional side view of a sensor configured with a pH sensor in the vicinity of a working electrode and a reference electrode, of an embodiment disclosed herein.

FIG. 3D is a top view of a sensor configured for measuring a hematocrit value according to an embodiment disclosed herein.

FIG. 3E is a top view of a sensor configured for measuring a hematocrit value according to an embodiment disclosed herein.

FIG. 4 is a side view of a multi-lumen catheter with a sensor assembly according to an embodiment disclosed and described herein.

FIG. 5 is a detail of the distal end of the multi-lumen catheter of FIG. 2 according to an embodiment disclosed and described herein.

FIG. 6 illustrates a hematocrit sensor according to an embodiment disclosed and described herein.

FIG. 7 is a side cross-sectional view of a sensor configured with a hematocrit sensor adaptable to a multi-lumen catheter according to an embodiment disclosed and described herein.

FIG. 8 is a flow chart illustrating a method of adjusting an analyte concentration value according to an embodiment disclosed and described herein.

FIG. 9 is a flow chart illustrating a method of adjusting an analyte concentration value according to an embodiment disclosed and described herein.

### DETAILED DESCRIPTION

Infusion sources, such as IV bag solutions, adapted for infusion and flushing of analyte sensors can vary widely in composition and pH. For example, some infusion sources may contain only saline solution while others may contain buffers, medications, or other components such as calibrants, resulting in infusion sources having a wide range of pH. For electrochemical sensors adapted to utilize enzyme electrodes to detect analyte, variations in the pH of the infusion source may affect the accuracy of the sensor's measurements.

The accuracy of the enzyme electrodes is affected by many factors, including pH. For example, enzyme reaction rates vary with pH. Enzymes are most active at an optimal pH and pH conditions below or above the optimal pH typically alter the enzyme's rate of reaction. Furthermore, some of the byproducts of enzyme driven reactions may also be affected by the internal local environmental pH of the electrochemical cell resulting in inaccurate analyte concentration values determined by the enzyme electrode sensor.

The enzymes used in electrochemical analyte sensors promote oxidation reactions that take place at the electroactive surface of the working electrode and produce an electro-active species, which may be measured as a change in current and correlated to the concentration of analyte in a sample. Changes in pH at or near the electroactive surface of the electrode affect the activity of enzymes and the concentration of byproducts produced by enzymatic reactions. The pH of the internal environment of an in vivo sensor may undergo, for example, change due to the influx of IV bag solutions, calibration solutions, or medications having a pH above 7.0 (basic) or below 7.0 (acidic). Furthermore, the pH of the sample being measured by the sensor, such as blood, can also vary. The blood pH in diabetic patients, for example, often fluctuates due to the increase or decrease of glucose in the bloodstream. Signals generated at certain pH values outside of a predetermined range of pH can produce inaccurate results. For example, the output signal from an enzyme-based glucose sensor may be significantly altered in a low pH environment than it would be under normal physiological pH conditions. In addition to pH, other factors such as the hematocrit level of blood, i.e., the percent or fraction of whole blood volume occupied by red blood cells, may also affect the accuracy of the enzyme electrode.

Thus, disclosed herein are analyte sensors and sensor assemblies comprising a membrane, at least one electrode disposed beneath the membrane, and either at least one pH sensor, disposed beneath the membrane and in close proximity to the at least one electrode, or a hematocrit sensor positioned in close proximity to the at least one electrode. More particularly, devices and methods for providing a correction factor for adjusting a glucose concentration value based on a measured pH value and/or a measured hematocrit level are disclosed. The various embodiments disclosed herein describe analyte sensors that measure analyte concentrations independent of the infusion source.

The following description and examples illustrate some exemplary embodiments of the disclosed invention in detail. Those of skill in the art will recognize that there may be numerous variations and modifications of this invention that may be encompassed by its scope. Accordingly, the description of a certain exemplary embodiment is not intended to limit the scope of the present invention.

### DEFINITIONS

In order to facilitate an understanding of the various aspects disclosed and described herein, the following are defined below.

The term “analyte” as used herein refers without limitation to a substance or chemical constituent of interest in a biological fluid (for example, blood) that may be analyzed. The analyte may be naturally present in the biological fluid, the analyte may be introduced into the body, or the analyte may be a metabolic product of a substance of interest or an enzymatically produced chemical reactant or chemical product of a substance of interest. Preferably, analytes include chemical entities capable of reacting with at least one enzyme and quantitatively yielding an electrochemically reactive product that is either amperometrically or voltammetrically detectable.

The phrases and terms “analyte measuring device,” “sensor,” and “sensor assembly” as used herein refer without limitation to an area of an analyte-monitoring device that enables the detection of at least one analyte. For example, the sensor may comprise a non-conductive portion, at least one working electrode, a reference electrode, and a counter electrode (optional), forming an electrochemically reactive surface at one location on the non-conductive portion and an electronic connection at another location on the non-conductive portion, and one or more layers over the electrochemically reactive surface.

The term “comprising” and its grammatical equivalents, as used herein is synonymous with “including,” “containing,” or “characterized by,” and is inclusive or open-ended and does not exclude additional, unrecited elements or method steps.

The term “subject” as used herein refers without limitation to mammals, particularly humans and domesticated animals.

The term “domain” as used herein refers without limitation to regions of a membrane that can be layers, uniform or non-uniform gradients (i.e., anisotropic) or provided as portions of the membrane.

The term “non-enzymatic” as used herein refers without limitation to a lack of enzyme activity. In some embodiments, a “non-enzymatic” membrane portion contains no enzyme; while in other embodiments, the “non-enzymatic” membrane portion contains inactive enzyme. In some embodiments, an enzyme solution containing inactive enzyme or no enzyme is applied.

The terms “inactive enzyme” or “inactivated enzyme” as used herein refers without limitation to an enzyme (e.g., glucose oxidase) that has been rendered inactive (e.g., “killed” or “dead”) and has no enzymatic activity. Enzymes can be inactivated using a variety of techniques known in the art, such as but not limited to heating, freeze-thaw, denaturing in organic solvent, acids or bases, cross-linking, genetically changing enzymatically critical amino acids, and the like. In some embodiments, a solution containing active enzyme can be applied to the sensor, and the applied enzyme subsequently inactivated by heating or treatment with an inactivating solvent.

The phrase “analyte concentration value” as used herein refers without limitation to a value corresponding to the amount of analyte per volume of a sample. For example, the analyte concentration value may be the amount of glucose present in a predetermined volume of bodily fluids of a subject, for example mg/dL.

The phrase “correction factor” as used herein refers without limitation to an amount of deviation in a measurement used to adjust the analyte concentration value. For example, a pH value corresponding to the pH of the electroactive portion of a glucose sensor may be used to calculate the amount of deviation in the glucose concentration value resulting from the effect of pH on the measurement. The calculated amount of deviation may then be used to adjust the measured glucose concentration value.

The term “algorithm” as used herein refers without limitation to a computational process (for example, programs) involved in transforming information from one state to another, for example, by using computer processing.

pH Sensor

In one aspect, the pH sensor essentially comprises an ion-sensitive electrode configuration. Ion-sensitive electrodes measure the activity of a specific ion, or ions, in a sample. In the case where the sample comprises bodily fluids, the ion activities typically measured are those of the hydrogen, sodium, potassium, and calcium cations (respectively H⁺ Na⁺, K⁺, and Ca²⁺). pH sensor are ion-sensitive electrodes that measure the concentration of H⁺ in a sample. Typically, the ion-sensitive electrode and a corresponding reference electrode are contacted with the sample. The ion-sensitive electrode may, in one instance, be constructed with an ion-exchanging membrane so that the potential difference between the ion-exchanging membrane and the sample is a function of the activity of a particular ion in the sample. The reference electrode is constructed so that the potential difference between the reference electrode and the sample is a constant, independent of the composition of the sample. By measuring the voltage across the ion-sensitive electrode and the reference electrode, the ion activity, and therefore the concentration, of a particular ion in the sample may be determined. Since the potential difference between the reference electrode and the sample is substantially constant and independent of pH, the potential difference between the pH sensor and the reference electrode, when immersed in the sample, varies linearly with pH at a given temperature according to the equation

$${V}_{p H} = {V}_{0} - \underline{kT ( ln 10 )} \cdot {e}^{( p H )}$$

where V₀ is and electrode-dependent constant, k is Boltzmann's constant, T is the temperature of the sample in degrees Kelvin, e is the charge of an electron and pH is the hydrogen ion concentration of the sample in pH units.

In another aspect, pH sensor include a conductor and an ion-sensitive membrane for sensing hydrogen ion concentration. For example, the pH sensor includes a conductor (e.g., a silver wire coated with silver chloride) immersed in an inner reference material, such as a weak hydrogen chloride solution having a known and constant pH, and an ion-sensitive glass membrane. The glass membrane permits the exchange of sodium ions in the glass for hydrogen ions in the sample. The result of this ion exchange is the development of a potential difference between the membrane and the sample which is related to the hydrogen ion activity in the sample.

Suitable ion-sensitive membranes may include glass membranes or may comprise polymeric membranes containing ionophores or hydrogen carriers such as tri-n-dodecylamine, 4-Nonadecylpyridine, N,N-Dioctadecylmethylamine, tribenzylamine, p-octadecyloxy-m-chlorophenylhydrazone mesoxalonitrile (OCPH), and hexabutyltriamindophosphate. For example, the ion-sensitive membrane may include polyvinyl chloride and tri-n-dodecylamine.

In yet another aspect, the pH sensor include a FET (field effect transistor configuration) such as a CHEMFET (chemical field effect transistor) or ISFET (ion-sensitive field effect transistor) or MOSFET (metal oxide semiconductor field effect transistor). pH measurements are based on the utilization of a change in gate potential of the ISFET device which results from the sensitivity thereof to the activity of H⁺ ions contained in the sample while a constant current or voltage is supplied to the source-to-drain passage of the ISFET device, with the resultant pH value being delivered from the source potential. In ISFET devices, the conductor normally applied to a gate insulating region of the field effect transistor is not utilized, and the gate insulating region is itself fabricated out of an ion-sensitive material. Suitable ion-sensitive materials for use in ISFET devices include silicon dioxide, silicon nitride, tantalum pentoxide, aluminum oxide, etc. Membranes containing enzymes can also be used as the ion-sensitive membrane in the ISFET pH sensor. For example, an ion-sensitive membrane containing immobilized glucose oxidase and a sodium salt can be used to measure the change in pH as glucose oxidase reacts with glucose to produce gluconic acid.

In one aspect, a pH sensor comprising an ion-sensitive membrane is provided. For example, the ion-sensitive membrane may include a glass membrane, a polymeric ion carrier membrane, metal oxide, enzyme-containing membrane or a combination of one or more of the foregoing membranes. In an exemplary embodiment, one or both of an active enzymatic portion or inactive-enzymatic or non-enzymatic portion of a membrane is deposited over a pH sensor. In other embodiments, an active enzymatic portion of a membrane is deposited over a hydrogen ion-sensitive membrane of a pH sensor. For example, the active enzymatic portion of a membrane may be deposited on a glass membrane of a miniature glass electrode or ISFET pH sensor. In one aspect, the pH sensor includes a field effect transistor. Other pH sensors can be employed, such as optical-based pH sensors.

### Sensor System and Sensor Assembly

The aspects disclosed and described herein disclosed relate to the use of an analyte sensor system that measures a concentration of analyte of interest or a substance indicative of the concentration or presence of the analyte. The sensor system is a continuous device, and may be used, for example, as or part of a subcutaneous, transdermal (e.g., transcutaneous), or intravascular device. The analyte sensor may use an enzymatic, chemical, electrochemical, or combination of such methods for analyte-sensing. The output signal is typically a raw signal that is used to provide a useful value of the analyte of interest to a user, such as a patient or physician, who may be using the device. In one aspect, a constant potential to the working and reference electrodes is applied to determine a current value. The current that is produced at the working electrode (and flows through the circuitry to the counter electrode) is substantially proportional to the amount of H₂O₂ that diffuses to the working electrode. For an enzymatic electrode sensor, the H2O2 is proportional to the amount of glucose present in the sample, therefore, a raw signal can be produced that is representative of the concentration of glucose in the user's body, and therefore can be utilized to estimate a meaningful glucose value, such as is described herein. Appropriate smoothing, calibration, correcting, and evaluation methods may be applied to the raw signal.

In one embodiment, a correction factor compensates for the effect that pH has on the measurement of analyte when converting the raw signal to an analyte concentration value. pH measurements may be used to provide a correction factor to correct for inaccurate raw signal outputs. For example, a signal generated from a pH sensor that is representative of the pH value of the environment of the working electrode may be produced by the sensor. The pH value may be used, for example, in an algorithm to calculate a correction factor to adjust the measured glucose concentration value. For example, a pH correction curve is provided that is programmed into an algorithm to calibrate the sensor output signal at a fixed glucose concentration as a function of pH.

In one aspect, a pH sensor for measuring the pH of the area proximal to the electroactive surface of the working electrode is provided. In one aspect, the pH sensor is positioned in close proximity to the working electrode and/or reference electrode to measure the pH of the local environment of one or more electrodes. In one aspect, the pH sensor is positioned in close proximity to one or more working electrodes.

In one embodiment, alone or in combination with the pH correction described above, a correction factor compensates for the effect the hematocrit level of a sample has on the measurement of analyte when converting the raw signal to an analyte concentration value. Hematocrit level measurements may be used to provide a correction factor to correct for inaccurate raw signal outputs. For example, a signal generated from a hematocrit sensor that is representative of the hematocrit level of a sample being measured may be produced by the sensor. The hematocrit level may be used, for example, in an algorithm to calculate a correction factor to adjust the measured glucose concentration value.

In one aspect, a hematocrit sensor for measuring the hematocrit level of a sample is provided. In one aspect, the hematocrit sensor is positioned in proximity to the working electrode and/or reference electrode.

Enzyme electrode sensors typically comprise one or more membrane layers. The membrane layers can include one or more electrode layers, enzyme layers, interference layers, flux limiting layers and/or biocompatible layers. Certain membrane layers can comprise dual functionality, for example, interference blocking and flux limiting can be provided in a single layer. Electrode chemistry in proximity to the electrode surface can be localized by the membrane layer due to diffusion rates of ions and neutral species in and out of the membranes. The membrane chemistry will likely dictate the extent and the affect of the local environment of the electrode surface. In one aspect, a portion of a membrane covers at least a portion of the pH sensor. For example, the membrane layers covering the pH sensor may also be the same layers covering the working electrode and/or reference electrode and blank electrode. The membrane layers covering the pH sensor may also be different from the membrane layers covering the working electrode. In one aspect, the one or more layers covering at least a portion of the pH sensor are substantially absent active enzymes. In one aspect, the hematocrit sensor is disposed beneath a portion of the membrane. For example, the hematocrit sensor comprises two or more electrodes that may be positioned beneath the membrane and in contact with a sample beneath the membrane to measure the impedance of the sample correlating to a hematocrit level. The electrodes may be separated from the membrane by a space such that the membrane is not in direct contact with the surface of the electrodes. In this way, the hematocrit sensor measures the impedance value of the sample in contact with the working electrode without being encumbered by the membrane. In another aspect, the hematocrit sensor is not disposed beneath the membrane. For example, the working electrode and a blank electrode may be disposed beneath a portion of the membrane and the hematocrit sensor may be positioned in proximity to the working and blank electrode without being disposed beneath the membrane.

One exemplary embodiment described in detail below utilizes a medical device, such as a catheter, with a glucose sensor assembly. In one aspect, a medical device with an analyte sensor assembly is provided for inserting the catheter into a subject's vascular system. The medical device with the analyte sensor assembly may include an electronics unit associated with the sensor, and a receiver for receiving and/or processing sensor data. Although a few exemplary embodiments of continuous glucose sensors may be illustrated and described herein, it should be understood that the disclosed embodiments may be applicable to any device capable of substantially continual or substantially continuous measurement of a concentration of analyte of interest and for providing an output signal that is representative of the concentration of that analyte.

### Electrodes and Electroactive Surface

The electrode and/or the electroactive surface of the sensor or sensor assembly disclosed herein comprises a conductive material, such as platinum, platinum-iridium, palladium, graphite, gold, carbon, conductive polymer, alloys, ink or the like. Although the electrodes can be formed by a variety of manufacturing techniques (bulk metal processing, deposition of metal onto a substrate, or the like), it may be advantageous to form the electrodes from screen printing techniques using conductive and/or catalyzed inks. The conductive inks may be catalyzed with noble metals such as platinum and/or palladium.

In one aspect, the electrodes and/or the electroactive surfaces of the sensor or sensor assembly are formed on a flexible substrate, such as a flex circuit. In one aspect, a flex circuit is part of the sensor and comprises a substrate, conductive traces, and electrodes. In one aspect, the electrodes and pH sensor are disposed on the sensor substrate. The traces and electrodes may be masked and imaged onto the substrate, for example, using screen printing or ink deposition techniques. The traces and the electrodes, and the electroactive surface of the electrodes may be comprised of a conductive material, such as platinum, platinum-iridium, palladium, graphite, gold, carbon, conductive polymer, alloys, ink or the like.

In one aspect, a counter electrode is provided to balance the current generated by the species being measured at the working electrode. In the case of a glucose oxidase based glucose sensor, the species being measured at the working electrode is H₂O₂. Glucose oxidase catalyzes the conversion of oxygen and glucose to hydrogen peroxide and gluconate according to the following reaction: Glucose+O₂→Gluconate+H₂O₂. Oxidation of H₂O₂ by the working electrode is balanced by reduction of any oxygen present, or other reducible species at the counter electrode. The H₂O₂ produced from the glucose oxidase reaction reacts at the surface of working electrode and produces two protons (2H⁺), two electrons (2e⁻), and one oxygen molecule (O₂). The electrons produce a detectable electrical current corresponding to the concentration of glucose in a sample. The environmental pH at the reaction site may affect the rate of the catalytic reaction and thus, the concentration of the H₂O₂ produced.

In one aspect, the pH sensor is provided to measure the pH value of the sample being measured. For example, the pH sensor measures the pH of blood and/or other bodily fluids. In one aspect, the pH sensor is provided to measure the pH of an infusion source. For example, the pH sensor can measure the pH of an IV bag solution, calibrant fluid, flush fluid, or other fluid including drugs and/or anticoagulant. For example, the pH sensor measures intravascular blood and calibrant fluids present in the sensor assembly. In one aspect, a pH sensor measures the pH or pH change at or near the site where the enzyme driven reaction takes place (i.e., the electroactive surface). In one aspect, the pH sensor is positioned in close proximity to the working electrode. In this way, the pH in the in the internal working environment of the sensor can be determined.

In one aspect, additional electrodes may be included within the sensor or sensor assembly, for example, a three-electrode system (working, reference, and counter electrodes) and/or one or more additional working electrodes configured as a baseline subtracting electrode, or which is configured for measuring additional analytes. The two working electrodes may be positioned in close proximity to each other, and in close proximity to the reference electrode. For example, a multiple electrode system may be configured wherein a first working electrode is configured to measure a first signal comprising glucose and baseline and an additional working electrode substantially similar to the first working electrode without an enzyme disposed thereon is configured to measure a baseline signal consisting of baseline only. In this way, the baseline signal generated by the additional electrode may be subtracted from the signal of the first working electrode to produce a glucose-only signal substantially free of baseline fluctuations and/or electrochemically active interfering species.

In one aspect, the sensor comprises from 2 to 5 electrodes. The electrodes may include, for example, the counter electrode (CE), working electrode (WE1), reference electrode (RE), the pH sensor (PE), and optionally a second working electrode (WE2). In one aspect, the sensor will have at least a CE, RE, PE and WE1. In one aspect, the addition of a WE2 is used, which may further improve the accuracy of the sensor measurement. In one aspect, the addition of a second counter electrode (CE2) may be used, which may further improve the accuracy of the sensor measurement.

The electroactive surface may be treated prior to application of any of the subsequent layers. Surface treatments may include for example, chemical, plasma or laser treatment of at least a portion of the electroactive surface. By way of example, the electrodes may be chemically or covalently contacted with one or more adhesion promoting agents. Adhesion promoting agents may include for example, aminoalkylalkoxylsilanes, epoxyalkylalkoxylsilanes and the like. For example, one or more of the electrodes may be chemically or covalently contacted with a solution containing 3-glycidoxypropyltrimethoxysilane.

In some alternative embodiments, the exposed surface area of the working (and/or other) electrode may be increased by altering the cross-section of the electrode itself. Increasing the surface area of the working electrode may be advantageous in providing an increased signal responsive to the analyte concentration, which in turn may be helpful in improving the signal-to-noise ratio, for example. The cross-section of the working electrode may be defined by any regular or irregular, circular or non-circular configuration.

### Membrane System

In general, membrane systems of enzyme electrode sensors include one or more domains. The membrane system can be deposited on the exposed electroactive surfaces and the pH sensor using known thin film techniques (for example, vapor deposition, spraying, electro-depositing, dipping, and the like). In alternative embodiments, however, other vapor deposition processes (e.g., physical and/or chemical vapor deposition processes) can be useful for providing one or more of the insulating and/or membrane layers, including ultrasonic vapor deposition, electrostatic deposition, evaporative deposition, deposition by sputtering, pulsed laser deposition, high velocity oxygen fuel deposition, thermal evaporator deposition, electron beam evaporator deposition, deposition by reactive sputtering molecular beam epitaxy, atmospheric pressure chemical vapor deposition (CVD), atomic layer CVD, hot wire CVD, low-pressure CVD, microwave plasma-assisted CVD, plasma-enhanced CVD, rapid thermal CVD, remote plasma-enhanced CVD, and ultra-high vacuum CVD, for example. However, the membrane system can be disposed over (or deposited on) the electroactive surfaces using any known method, as will be appreciated by one skilled in the art.

In some embodiments, one or more domains of the membrane systems are formed from materials such as silicone, polytetrafluoroethylene, polyethylene-co-tetrafluoroethylene, ethylene vinyl acetate (EVA), polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, polysulfones and block copolymers thereof including, for example, di-block, tri-block, alternating, random and graft copolymers.

In one aspect, one or more membranes are provided on the sensor comprising an active enzymatic portion and inactive-enzymatic portion or non-enzymatic portion.

In one aspect, the active enzymatic portion of the membrane includes one or more membrane layers comprising a polymer (e.g., poly-N-vinylpyrrolidone) and an enzyme. The enzyme is preferably immobilized in the sensor. The enzyme may be encapsulated within the hydrophilic polymer and may be cross-linked or otherwise immobilized therein. The enzymatic portion may be deposited directly on at least a portion the electroactive surface of one or more working electrodes. The active enzymatic portion of the membrane may further include at least one protein and/or natural or synthetic material. For example, the active enzymatic portion of the membrane may further include, serum albumins, polyallylamines, polyamines and the like, as well as combination thereof.

In one aspect, the inactive-enzymatic portion or non-enzymatic portion of the membrane includes one or more layers that contain no enzymes or that comprise inactive enzymes. The inactive-enzymatic portion or non-enzymatic portion of the membrane may include, for example, an interference layer or a flux limiting membrane.

### Hematocrit Detection and Correction

Other factors, such as hematocrit, can affect the output of the raw signal generated from a sensor. Hematocrit is the percent or fraction of whole blood volume occupied by red blood cells, which may vary from about 0.2 for individuals who suffer from anemia to about 0.6 for newborns. While not to be held to any particular theory, it is generally believed that hematocrit interferes with the detections of glucose through a volume exclusion effect. For example, for a given volume of blood, the greater the hematocrit, the lower the relative volume of blood plasma and the less glucose is available for the glucose-oxidase reaction. Thus, hematocrit tends to cause an artificially high glucose concentration for low hematocrit levels and, conversely, an artificially low glucose concentration for high hematocrit levels. This “hematocrit effect” can be deleterious to the accuracy of an analyte sensor intended for use in the circulatory system, for example in the intravenous environment. Such an analyte sensor has been proposed for achieving Tight Glycemic Control (TGC) within an operating room (OR) or intensive care unit (ICU) environment. In such use environments, hematocrit levels are routinely measured frequently. However, when a patient transitions to the general ward of a hospital, the frequency of hematocrit measurement is relatively lower than that of either the OR or ICU. Thus, in one embodiment measuring hematocrit levels and adjusting determined glucose concentration values in real time is provided.

In another embodiment, alone or in combination with the above pH correction, algorithms are provided that compensate for hematocrit levels when converting the raw signal to analyte concentration values. For example, a first correction factor associated with the pH of the environment about one or more of the working electrodes can be used in combination with a second correction factor associated with a hematocrit value of the environment about one or more of the working electrodes. The pH measured can be of the sample being measured or of an infusion fluid presented to the analyte sensor, such as a calibrant fluid, flush fluid, or other fluid including drugs and/or anticoagulant.

The impedance or conductivity (the reciprocal of resistance) of whole blood is dependent on hematocrit. In one aspect, the impedance value of a blood sample between two electrodes is measured to determine the hematocrit of a blood sample and therefore to correct for the hematocrit interference of the determined glucose concentration value. For example, two electrodes can be used to measure the conductivity (i.e., the reciprocal of impedance Zb) of a blood sample applied to two electrodes. Two or more electrodes can, for example, be positioned on opposite sides of a column of blood or in the path of a flowing channel of blood. In an exemplary embodiment, an oscillator applies an alternating voltage to two electrodes and the resulting voltage drop across the sample positioned between the two electrodes is measured and converted to a signal. The signal is proportional to the conductivity or reciprocal impedance of the sample and can be correlated to a hematocrit value using a calibration curve. In another exemplary embodiment, two electrodes apply a current to a sample and two electrodes measure the voltage that is produced across the tissue by the current to determine impedance (V/I). The hematocrit value may be used, for example, to adjust a measured analyte concentration. In one embodiment, a signal correlating to the hematocrit value is used to calculate a corrective factor for adjusting a measured analyte concentration. In another embodiment, an algorithm determines the corrective factor. Since the impedance or conductivity of whole blood is dependent on hematocrit, for a glucose sensor, for example, having an electrode or sensing wire dedicated to impedance or conductivity measurements of whole blood could provide a signal to an algorithm containing control box of the sensor system which would adjust the glucose concentration for the hematocrit level in real time.

In one aspect, optical properties of light passing through a blood sample are measure to determine hematocrit levels of a blood sample. In one exemplary embodiment, one or more optical fibers transmit light from one or more light sources through a blood sample at a specific wavelength or wavelengths and the light absorbed, transmitted, or scattered is measured by a light detector to derive the hematocrit level of the sample. The transmission of light though red blood cells is complicated by scattering components from plasma. An algorithm based on optical spectra with known hematocrit values, algorithms incorporating scattering coefficients and molecular extinction, or measurements of scattering at specific wavelengths, for example, may be used to correct measured absorbance/transmission values in order to determine the hematocrit level in a blood sample. The hematocrit value may be used, for example, to adjust a measured analyte concentration. In one embodiment, a signal correlating to the hematocrit value is used to calculate a corrective factor for adjusting a measured analyte concentration. For example, the output of an optical hematocrit measurement could be sent to an algorithm containing control box of the sensor system which would adjust the glucose concentration for the hematocrit level in real time.

In one embodiment, a hematocrit sensor is positioned in proximity to at least one electrode disposed beneath a membrane. The hematocrit sensor may comprise, for example, one or more electrodes or one or more optical fibers. In one embodiment, the electrodes of the hematocrit sensor are disposed on a substrate. In other embodiments, the hematocrit sensor is disposed on one surface of the substrate and the at least one electrode is positioned on the opposing surface of the substrate. In another embodiment, the hematocrit sensor is disposed beneath an active enzymatic portion and/or an inactive-enzymatic or non-enzymatic portion of the membrane.

### Bioactive Agents

In some alternative embodiments, a bioactive agent may be optionally incorporated into the above described sensor system, such that the bioactive agent diffuses out into the biological environment adjacent to the sensor. Additionally or alternately, a bioactive agent may be administered locally at the exit-site or implantation-site. Suitable bioactive agents include those that modify the subject's tissue response to any of the sensor or components thereof. For example, bioactive agents may be selected from anti-inflammatory agents, anti-infective agents, anesthetics, inflammatory agents, growth factors, immunosuppressive agents, antiplatelet agents, anti-coagulants, anti-proliferates, ACE inhibitors, cytotoxic agents, anti-bather cell compounds, vascularization-inducing compounds, anti-sense molecules, or mixtures thereof.

### Sensor Assembly Adapted for Intravenous Insertion

In one aspect, an electrochemical analyte sensor assembly may be configured for an intravenous insertion to a vascular system of a subject. In order to accommodate the sensor within the confined space of a device suitable for intravenous insertion, the sensor assembly may comprise a flexible substrate, such as a flex circuit. For example, the flexible substrate of the flex circuit may be configured as thin conductive electrodes coated on a non-conductive material such as a thermoplastic or thermoset. Conductive traces may be formed on the non-conductive material and electrically coupled to the thin conductive electrodes. The electrodes of the flex circuit may be as described above wherein the traces and contacts of flex circuit supports and electrically couples to the electrodes. In other embodiments, the sensor assembly may comprise a plurality of wires. For example, the plurality of wires may be juxtaposed and coated or adhered together with an insulating material.

The sensor assembly may comprise at least one reference electrode and at least one working electrode, the at least one working electrode having an electroactive surface capable of providing a detectable electrical output upon interaction with an electrochemically detectable species. The sensor assembly may further comprise at least one counter electrode. In one aspect, the sensor assembly contains at least one reference electrode, at least one working electrode, and at least one pH sensor. In one aspect, the sensor assembly contains at least one blank electrode, at least one working electrode, and a hematocrit sensor. In one aspect, the sensor assembly contains two or more working electrodes, and two or more counter electrodes. In one aspect, the flex circuit contains two or more working electrodes, two or more pH sensor, two or more blank electrodes, and two or more counter electrodes.

At least one working electrode, at least one pH sensor, and at least one reference or blank electrode may be disposed beneath a portion of the membrane. The active enzymatic portion of the membrane may be in contact with at least a portion of the electroactive surface of the working electrode. The pH sensor may be disposed beneath the active enzymatic portion and/or inactive-enzymatic or non-enzymatic portions of the electrode. In one embodiment, the at least one pH sensor is not disposed beneath a portion of the membrane. For example, the pH sensor may be positioned in close proximity to a working electrode without being disposed beneath the enzymatic or inactive or non-enzymatic portions of the membrane. The reference electrode may be disposed beneath the active enzymatic portion and/or inactive-enzymatic or non-enzymatic portions of the electrode. In other embodiments, at least one working electrode, a hematocrit sensor, and at least one blank or reference electrode may be disposed beneath a portion of the membrane. The hematocrit sensor may be disposed beneath the active enzymatic portion and/or inactive-enzymatic or non-enzymatic portions of the electrode. For example, the hematocrit sensor may be positioned in close proximity to a working electrode without being disposed beneath the enzymatic or inactive or non-enzymatic portions of the membrane. In other embodiments, the hematocrit sensor is not disposed beneath a portion of the membrane. The flex circuit preferably is configured to be electrically configurable to a control unit. An example of an electrode of a flex circuit and it construction is found in co-assigned U.S. Application Nos. 2007/0202672 and 2007/0200254, incorporated herein by reference in their entirety.

Medical devices adaptable to the sensor assembly as described above include, but are not limited to a central venous catheter (CVC), a pulmonary artery catheter (PAC), a probe for insertion through a CVC or PAC or through a peripheral IV catheter, a peripherally inserted catheter (PICC), Swan-Ganz catheter, an introducer or an attachment to a Venous Arterial blood Management Protection (VAMP) system. Any size/type of Central Venous Catheter (CVC) or intravenous devices may be used or adapted for use with the sensor assembly.

For the foregoing discussion, the implementation of the sensor or sensor assembly is disclosed as being placed within a catheter; however, other devices as described above are envisaged and incorporated in aspects disclosed and described herein. The sensor assembly will preferably be applied to the catheter so as to be flush with the OD of the catheter tubing or the sensor may be recessed. This may be accomplished, for example, by thermally deforming or skiving the OD of the tubing to provide a recess for the sensor. The sensor assembly may be bonded in place, and sealed with an adhesive (i.e. urethane, 2-part epoxy, acrylic, etc.) that will resist bending/peeling, and adhere to the urethane CVC tubing, as well as the materials of the sensor. Small diameter electrical wires may be attached to the sensor assembly by soldering, resistance welding, or conductive epoxy. These wires may travel from the proximal end of the sensor, through one of the catheter lumens, and then to the proximal end of the catheter. At this point, the wires may be connected to an electrical connector, for example by solder or by ribbon cable with suitable connectors.

The sensor assembly as disclosed herein can be added to a catheter in a variety of ways. For example, an opening may be provided in the catheter body and a sensor or sensor assembly may be mounted inside the lumen at the opening so that the sensor would have direct blood contact. In one aspect, the sensor or sensor assembly may be positioned proximal to all the infusion ports of the catheter. In this configuration, the sensor would be prevented from or minimized in measuring otherwise detectable infusate concentration instead of the blood concentration of the analyte. Another aspect, an attachment method may be an indentation on the outside of the catheter body and to secure the sensor inside the indentation. This may have the added advantage of partially isolating the sensor from the temperature effects of any added infusate. Each end of the recess may have a skived opening to 1) secure the distal end of the sensor and 2) allow the lumen to carry the sensor wires to the connector at the proximal end of the catheter.

Preferably, the location of the sensor assembly in the catheter will be proximal (upstream) of any infusion ports to prevent or minimize IV solutions from affecting analyte measurements. In one aspect, the sensor assembly may be about 2.0 mm or more proximal to any of the infusion ports of the catheter.

In another aspect, the sensor assembly may be configured such that flushing of the catheter (i.e. saline solution) may be employed in order to allow the sensor assembly to be cleared of any material that may interfere with its function.

### Sterilization of the Sensor or Sensor Assembly

Generally, the sensor or the sensor assembly as well as the device that the sensor is adapted to are sterilized before use, for example, in a subject. Sterilization may be achieved using radiation (e.g., electron beam or gamma radiation) or flash-UV sterilization, or other high energy radiation sterilization means known in the art.

Disposable portions, if any, of the sensor, sensor assembly or devices adapted to receive and contain the sensor preferably will be sterilized, for example using e-beam or gamma radiation or other know methods. The fully assembled device or any of the disposable components may be packaged inside a sealed non-breathable container or pouch.

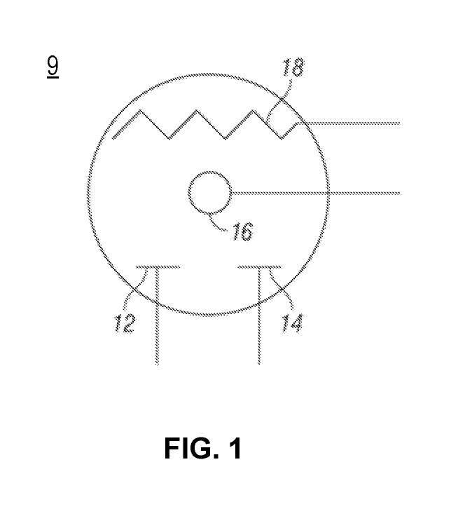

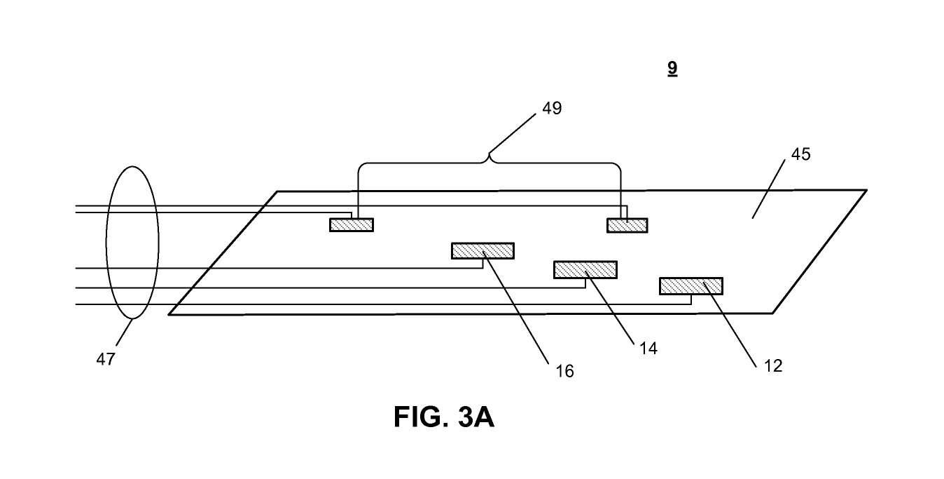

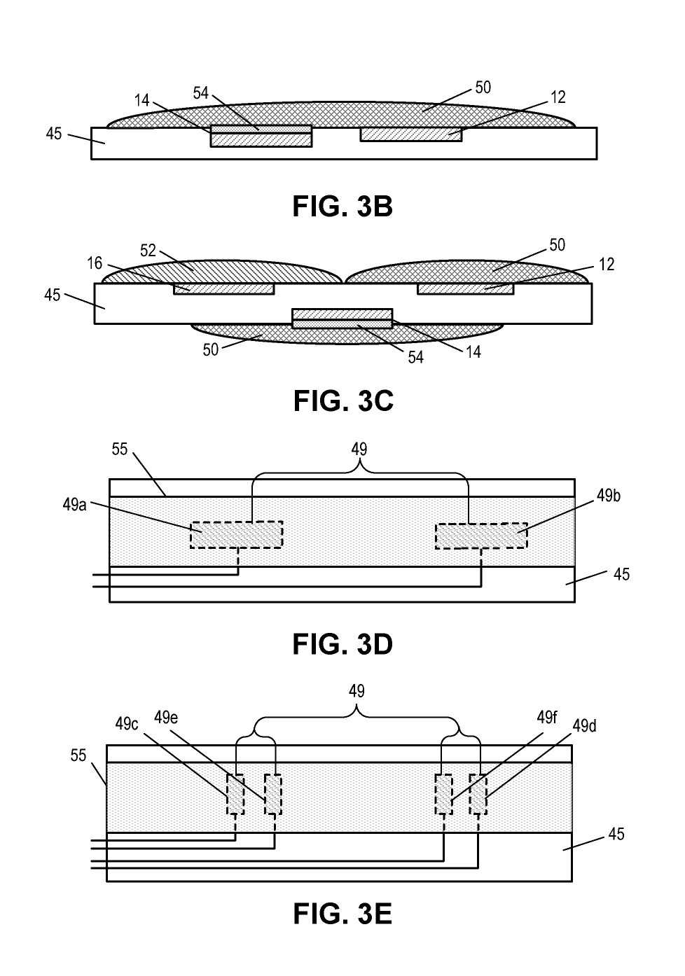

Referring now to the Figures, FIG. 1 is a schematic diagram of an amperometric, four-electrode sensor 9. In the illustrated embodiment, the sensor 9 includes a working electrode 12 and a pH sensor 14. The working electrode 12 may be a platinum based enzyme electrode, i.e. an electrode containing or immobilizing an enzyme layer. In one embodiment, the working electrode 12 may immobilize an oxidase enzyme. In some embodiments, the sensor is a glucose sensor, in which case the working electrode 12 may immobilize a glucose oxidase enzyme. The working electrode 12 may be formed using platinum, or a combination of platinum and graphite materials. The pH sensor 14 is discussed in more detail below regarding FIGS. 3A-3C. The sensor 9 further includes a reference electrode 16 and a counter electrode (not shown). The reference 16 can function as a counter electrode, or a reference electrode. In some aspects, a counter electrode and a reference electrode are employed in the instant disclosure. In an exemplary embodiment, the reference electrode 16 establishes a fixed potential from which the potential of the counter electrode and the working electrode 12 or the pH sensor 14 can be established. In other embodiments, the reference 16 functions as a blank electrode. In some embodiments, the sensor 9 comprises the working electrode 12, the reference electrode 16, and a hematocrit sensor comprising two or more electrodes or at least one optical fiber. The sensor 9 may additionally include one or more electrodes such as electrodes associated with a hematocrit sensor or another reference electrode for use in connection with the pH sensor 14. The counter electrode 18 provides a working area for conducting the majority of electrons produced from the oxidation chemistry back to the blood solution. During normal operation, the counter prevents excessive current from passing through the reference and working electrodes that may reduce their service life. However, the counter electrode may not typically have capacity to reduce current surges caused by spikes, which may affect the electrodes.

The amperometric sensor 9 operates according to an amperometric measurement principle, where the working electrode 12 is held at a positive potential relative to the reference electrode/counter 16. In one embodiment of a glucose monitoring system, the positive potential is sufficient to sustain an oxidation reaction of hydrogen peroxide, which is the result of glucose reaction with glucose oxidase. Thus, the working electrode 12 may function as an anode, collecting electrons produced at its surface that result from the oxidation reaction. The collected electrons flow into the working electrode 12 as an electrical current. In one embodiment with the working electrode 12 coated with glucose oxidase, the oxidation of glucose produces a hydrogen peroxide molecule for every molecule of glucose when the working electrode 12 is held at a potential between about +350 mV and +850 mV. For example, the working electrode 12 can be held at a potential between about +450 mV and about +750 mV. The hydrogen peroxide produced oxidizes at the surface of the working electrode 12 according to the equation:

H₂O₂→2H⁺+O₂+2e⁻

The equation indicates that two electrons are produced for every hydrogen peroxide molecule oxidized. Thus, under certain conditions, the amount of electrical current may be proportional to the hydrogen peroxide concentration. Since one hydrogen peroxide molecule is produced for every glucose molecule oxidized at the working electrode 12, a linear relationship exists between the blood glucose concentration and the resulting electrical current. The embodiment described above demonstrates how the working electrode 12 may operate by promoting anodic oxidation of hydrogen peroxide at its surface. Other embodiments are possible, however, wherein the working electrode 12 may be held at a negative potential. In this case, the electrical current produced at the working electrode 12 may result from the reduction of oxygen. The following article provides additional information on electronic sensing theory for amperometric glucose biosensors: J. Wang, “Glucose Biosensors: 40 Years of Advances and Challenges,” Electroanaylsis, Vol. 13, No. 12, pp. 983-988 (2001).

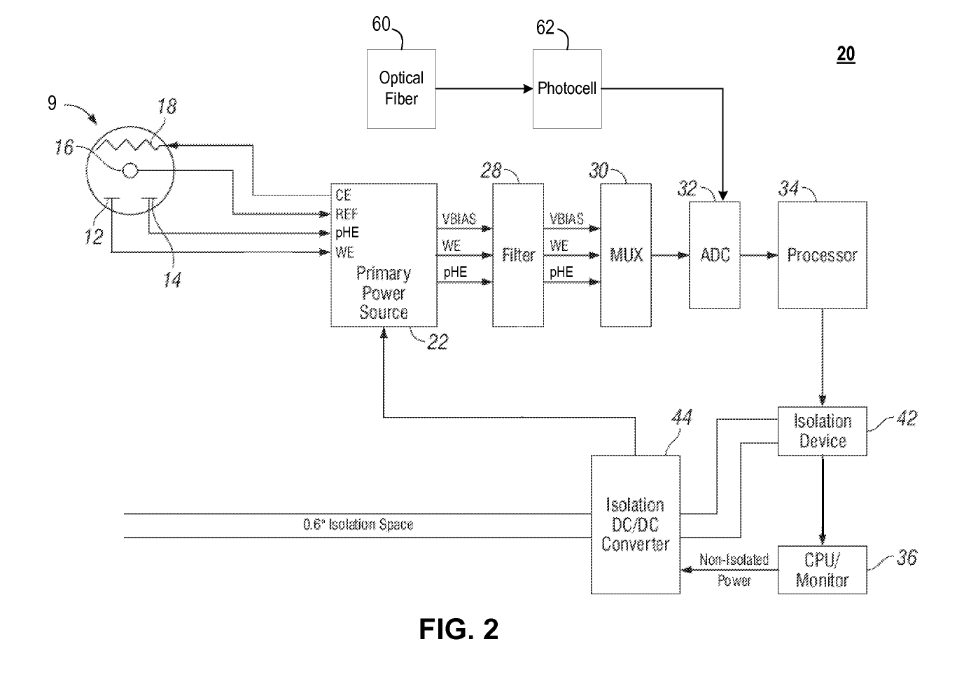

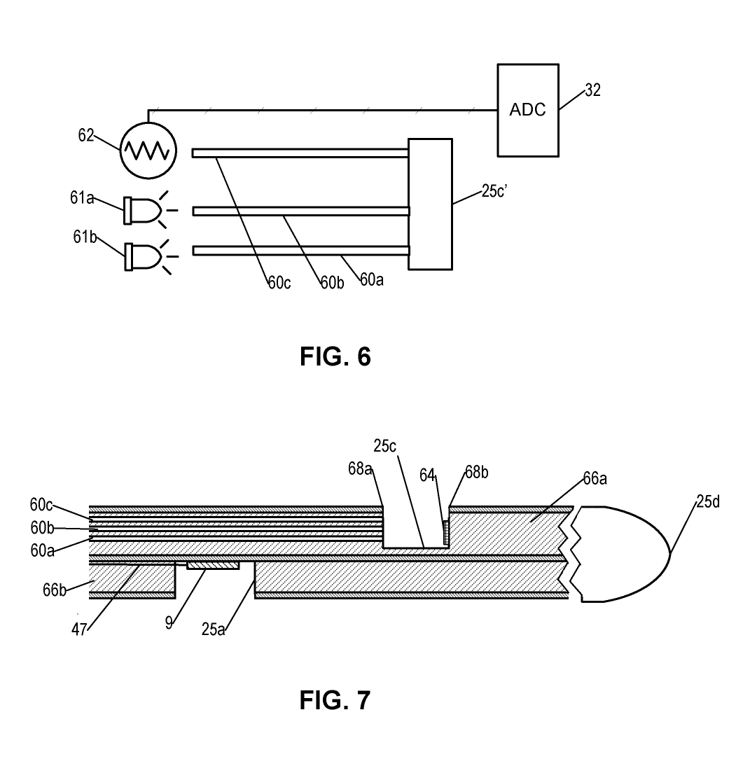

FIG. 2 illustrates a schematic block diagram of a system 20 for operating an electro-chemical sensor such as an amperometric or potentiometric sensor, such as a glucose sensor. In particular, FIG. 2 discloses a system comprising an amperometric sensor. In addition, the illustrated embodiments also shows an optical fiber 60 transmitting light to a photocell 62 for measuring hematocrit levels as described in more detail below with regard to FIGS. 6-7. In some embodiments, the system 200 includes either a hematocrit sensor or a pH sensor. For example, the system 200 may include the pH sensor 14, but not the optical fiber 60 and photocell 62 or the system 200 may include a hematocrit sensor comprising the optical fiber 60 or two or more electrodes, but not the pH sensor 14. As more fully disclosed in U.S. patent application Ser. No. 11/696,675, filed Apr. 4, 2007, and titled ISOLATED INTRAVENOUS ANALYTE MONITORING SYSTEM, a typical system for operating an amperometric sensor includes a potentiostat 22 in communication with the sensor 9. In normal operation, the potentiostat both biases the electrodes of the sensor and provides outputs regarding operation of the sensor. As illustrated in FIG. 2, the potentiostat 22 receives signals WE, pHE, and REF respectively from the working electrode 12, pH electrode 14, and the reference electrode 16. The potentiostat further provides a bias voltage CE input to the counter electrode 18. The potentiostat 22, in turn, outputs the signals WE, pHE from the working electrode 12 and pH sensor 14 and a signal representing the voltage potential VBIAS between the counter electrode 18 and the reference electrode 16.

A potentiostat is a controller and measuring device that, in an electrolytic cell, keeps the potential of the working electrode 12 at a constant level with respect to the reference electrode 16. It consists of an electric circuit which controls the potential across the cell by sensing changes in its electrical resistance and varying accordingly the electric current supplied to the system: a higher resistance will result in a decreased current, while a lower resistance will result in an increased current, in order to keep the voltage constant.

Another function of the potentiostat is receiving electrical current signals from the working electrode 12 or pH sensor 14 for output to a controller. As the potentiostat 22 works to maintain a constant voltage for the working electrode 12 or pH sensor 14, current flow through the working electrode 12 or pH sensor 14 may change. The current signals of the working electrode 12 indicate the presence of an analyte of interest in an intravenous sample such as blood. The current signals of the pH sensor 14 indicate a pH value of an intravenous sample or of an infusion source. In addition, the potentiostat 22 holds the counter electrode 18 at a voltage level with respect to the reference electrode 16 to provide a return path for the electrical current to the bloodstream, such that the returning current balances the sum of currents drawn in the working electrode 12.

While a potentiostat is disclosed herein as the first or primary power source for the electrolytic cell and data acquisition device, it must be understood that other devices for performing the same functions may be employed in the system and a potentiostat is only one example. For example, an amperostat, sometimes referred to as a galvanostat, can be used.

As is illustrated in FIG. 2, the output of the potentiostat 22 is typically provided to a filter 28, which removes at least some of the spurious signal noise caused by either the electronics of the sensor or control circuit and/or external environmental noise. The filter 28 is typically a low pass filter, but can be any type of filter to achieve desired noise reduction.

In FIG. 2, a multiplexer 30 may be employed to transfer the signals from the potentiostat 22, namely 1) the signals WE, pHE from the working electrode 12 and pH sensor 14; and 2) the bias signal VBIAS representing the voltage potential between the counter electrode 18 and the reference electrode 16 to the processor 34. The signals are also provided to an analog to digital converter (ADC) 32 to digitize the signals prior to input to the processor. Signals from a photocell 62 that measures the transmittance/absorbance or scattering of light through a sample from optical fibers 60 are also provided to ADC 32 as is described in more detail below with regard to FIG. 6.

The processor uses algorithms in the form of either computer program code where the processor is a microprocessor or transistor circuit networks where the processor is an application-specific integrated circuit (ASIC) or other specialized processing device to determine the amount of analyte in a substance, such as the amount of glucose in blood. The results determined by the processor may be provided to a monitor or other display device 36. As illustrated in FIG. 2 and more fully described in U.S. patent application Ser. No. 11/696,675, filed Apr. 4, 2007, and titled ISOLATED INTRAVENOUS ANALYTE MONITORING SYSTEM, the system may employ various devices to isolate the sensor 9 and associated electronics from environmental noise. For example, the system may include an isolation device 42, such as an optical transmitter for transmitting signals from the processor to the monitor 36 to avoid backfeed of electrical noise from the monitor 36 to the sensor and its associated circuitry. Additionally, an isolated main power supply 44 for supplying power to the circuit, such as an isolation DC/DC converter is provided.

FIG. 3A is the amperometric sensor 9 in the form of a flex circuit that incorporates a sensor embodiment disclosed herein. While a flex circuit assembly is depicted, it is intended that the embodiments disclosed herein are generally applicable to other configurations, such as dual wire electrodes and the like. Thus, sensor 9 formed on a substrate 45 (e.g., a flex substrate, such as copper foil laminated with polyimide) comprises the working electrode 12, the pH sensor 14, the hematocrit sensor 49, and the reference electrode 16, which may function as a reference, blank, or counter electrode, referred to herein as the reference electrode 16. In other embodiments, sensor 9 includes at least one electrode or at least two electrodes and either the hematocrit sensor 49 or the pH sensor 14. In another embodiment, one or more additional working electrodes or may be included on the substrate 45. A membrane system is preferably deposited over working electrode 12, pH sensor 14, and reference electrode 16, such as described in more detail with reference to FIGS. 3B and 3C below. A membrane system may also be deposited over hematocrit sensor 49. Electrical wires 47 transmit power to the electrodes for sustaining an oxidation or reduction reaction, and may also carry signal currents to a detection circuit (not shown) indicative of a parameter being measured. The parameter being measured may be any analyte of interest that occurs in, or may be derived from, blood chemistry. In one embodiment, the analyte of interest is hydrogen peroxide, formed from reaction of glucose with glucose oxidase, thus having a concentration that is proportional to blood glucose concentration.

FIG. 3B depicts a cross-sectional side view of a portion of substrate 45 in the vicinity of the working electrode 12 and pH sensor 14 of an embodiment disclosed herein. In some embodiments, the sensor 9 includes two or more pH sensors. Sensor 9 includes a sensor membrane comprising an active enzymatic portion 50. Additional membrane layers can be positioned between active enzymatic portion 50 and the electrodes, for example, electrode layers. The working electrode 12 may be at least partially coated with active enzymatic portion 50. Active enzymatic portion 50 is selected to chemically react when the sensor is exposed to certain reactants, for example, found in the bloodstream. For example, in an embodiment for a glucose sensor, active enzymatic portion 50 may contain glucose oxidase, such as may be derived from Aspergillus niger (EC 1.1.3.4), type II or type VII.

The exposed electroactive portion of working electrode 12 is configured to measure the concentration of an analyte. In an enzymatic electrochemical sensor for detecting glucose, for example, the working electrode measures the hydrogen peroxide produced by an enzyme catalyzed reaction of the analyte being detected and creates a measurable electronic current. The measured current or output signal may used to calculate the concentration of glucose in the blood using an algorithm. The algorithm may include, for example, additional correcting calculations from other measurements. In some embodiments, pH sensor 14 measure a pH value at or near the electroactive portion of working electrode 12. The measured pH value may be used in algorithm or a pH correction curve to calculate a corrected glucose concentration.

In some embodiments, pH sensor 14 is positioned in close proximity to working electrode 12. pH sensor 14 may be positioned, for example, close to working electrode 12 so that pH measurements can be taken in the area immediately surrounding the working electrode. pH sensor 14 may be positioned, for example, at a predetermined distance from working electrode 12 and reference electrode 16. For example, pH sensor 14 can be positioned in closer proximity to working electrode 12 than reference electrode 16. pH sensor 14 can also be, for example, be positioned at an equal distance from working electrode 12 and reference electrode 16. In the illustrated embodiment of FIG. 3B, pH sensor 14 and working electrode 12 are both disposed underneath the active enzymatic portion 50. In other embodiments, pH sensor 4 is disposed beneath an inactive-enzymatic or non-enzymatic membrane 52.

As discussed above, suitable pH sensor include, for example, ion-selective field effect transistors (ISFET) devices, pH sensitive polymeric electrodes, miniature glass electrodes, fiber optic pH probes, or any other pH device. In one embodiment, the pH sensor 14 comprises an ion-sensitive membrane 54, such as a hydrogen ion-sensitive membrane. In the illustrated embodiment, the pH sensor 14 is disposed beneath the ion-sensitive membrane 54. The ion-sensitive membrane 54 may be disposed beneath the active enzymatic portion 50 and/or the inactive-enzymatic or non-enzymatic portion 52. In one embodiment, the ion-sensitive membrane is in contact with at least a portion of one or both of the active enzymatic portion 50 and inactive-enzymatic or non-enzymatic portion 52. In other embodiments, the pH sensor 14 is not disposed beneath the active enzymatic portion 50 and/or inactive-enzymatic or non-enzymatic portion 52. The ion-sensitive membrane 54 is a membrane associated with pH sensing such as a glass membrane or resin material, a polymer containing hydrogen carriers, a metal oxide, or any other ion-sensitive coating for use in measuring a pH value. In some embodiments, the active enzymatic portion 50 or inactive-enzymatic or non-enzymatic portion 52 is sensitive to hydrogen ions. For example, active enzymatic portion 50 may be deposited over a source component and drain component of an ISFET pH sensor. The active enzymatic portion 50 may contain a compound that interacts with hydrogen ions, such as a hydrogen carrier. The interaction of the active enzymatic portion 50 with hydrogen ions results in a detectable current flow between the source and the drain for measuring a pH value. In this way, active enzymatic portion 50 itself acts as a hydrogen ion sensitive-membrane and the ion-sensitive membrane 54 is optional. In some embodiments, only the active enzymatic portion 50 or inactive-enzymatic or non-enzymatic portion 52 are disposed on the pH sensor 14. The pH sensor 14 may also include an internal reference electrode, internal reference materials, a FET, etc.

FIG. 3C depicts a cross-sectional side view of an alternative sensor embodiment comprising electrodes on opposite sides of a substrate, in the vicinity of the working electrode 12, pH sensor 14, and reference electrode 16, with a partitioned membrane over the working and reference electrodes, respectively. Working electrode 12 and pH sensor 14 disposed on opposing surfaces of the flex circuit are shown at least partially coated with the active enzymatic portion 50. The sensor membrane is partitioned into the active enzymatic portion 50 and the inactive-enzymatic or non-enzymatic portion 52. Reference electrode/counter 16 is shown disposed beneath inactive-enzymatic or non-enzymatic portion 52. In some embodiments, the pH sensor 14 is disposed beneath the inactive-enzymatic or non-enzymatic portion 52 in proximity to the reference electrode 16. The arrangement of partitioned membranes depicted in FIG. 3C can be utilized in a dual wire electrode configuration. For example, inactive-enzymatic or non-enzymatic portion 52 can be disposed on blank wire electrode while active enzymatic portion 50 can be disposed on working wire electrode and pH sensor.

In the illustrated embodiment, reference electrode 16 and working electrode 12 are disposed on one surface of substrate 45 and pH sensor 14 is disposed on the opposing surface. In other exemplary embodiments, pH sensor 14 is disposed on the same surface of substrate 45 as working electrode 12. In still other exemplary embodiments, pH sensor 14 is disposed on the same surface of substrate 45 as reference electrode 16. In still other embodiments, pH sensor 14, working electrode 12, and reference electrode 16 are disposed on the same surface of substrate 45 as shown in FIG. 3A.

FIGS. 3D-3E each depict a top view of an alternative sensor embodiment comprising sample 55 applied to hematocrit sensor 49 for measuring a signal corresponding to a hematocrit value of sample 55. In the illustrated embodiment, sample 55 is bodily fluids, such as blood. In some embodiments, the electrodes used to measure hematocrit comprise two or more electrodes. In other embodiments, the hematocrit sensor 49 is positioned on one surface of the substrate 45 and at least one electrode is positioned on the opposing surface of the substrate. For example, the working electrode 12 and/or the reference electrode 16 may be positioned on one surface of the substrate and the electrodes 49a and 49b may be positioned on the opposing surface of the substrate. In other embodiments, the electrodes 49a and 49b are disposed beneath the active enzymatic portion 50 or inactive-enzymatic or non-enzymatic portion 52 of the membrane. Electrodes 49a, 49b may include, for example, working electrode 12, reference electrode 16, or two or more separate electrodes. In FIG. 3D, electrodes 49a, 49b are connected to an oscillator (not shown) which applies an alternating voltage to the electrodes in contact with sample 55. The voltage drop across sample 55 is measured and converted to a signal. In some embodiments, the signal is dependent on the impedance of sample 55 and is correlated to a hematocrit value using a calibration curve. The signal may also be used, for example, to calculate a correction factor for adjusting a measured analyte concentration value. In FIG. 3E, hematcrit sensor 49 comprise a set of four electrodes including two electrodes 49c, 49d for applying current to the sample 55 and two electrodes 49e, 49f for measuring voltage across the sample to provide a signal corresponding to the impedance of the sample 55. In the illustrated embodiment, the voltage measuring electrode 49e, 49f are positioned between the current applying electrodes 49c, 49d. The signal can be correlated to the hematocrit level of the sample and can be used to determine a correction factor to adjust a measured analyte concentration value.

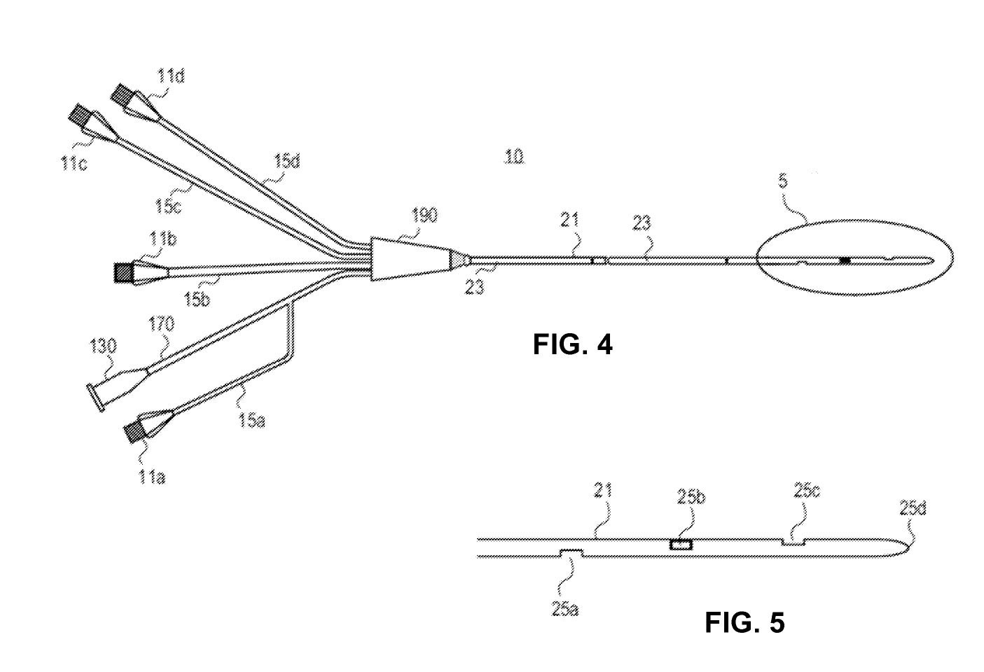

Referring now to FIGS. 4-5, aspects of the sensor adapted to a central line catheter with a sensor or sensor assembly are discussed as exemplary embodiments, without limitation to any particular intravenous device. FIG. 4 shows a sensor assembly within a multi-lumen catheter. The catheter assembly 10 may include multiple infusion ports 11a, 11b, 11c, 11d and one or more electrical connectors 130 at its most proximal end. A lumen 15a, 15b, 15c, or 15d may connect each infusion port 11a, 11b, 11c, or 11d, respectively, to a junction 190. Similarly, the conduit 170 may connect an electrical connector 130 to the junction 190, and may terminate at junction 190, or at one of the lumens 15a-15d (as shown). Although the particular embodiment shown in FIG. 4 is a multi-lumen catheter with an electrical connector, other embodiments having other combinations of lumens and connectors are possible, including a single lumen catheter, a catheter having multiple electrical connectors, etc. In another embodiment, one of the lumens and the electrical connector may be reserved for a probe or other sensor mounting device, or one of the lumens may be open at its proximal end and designated for insertion of the probe or sensor mounting device.

The distal end of the catheter assembly 10 is shown in greater detail in FIG. 5. At one or more intermediate locations along the distal end, the tube 21 may define one or more ports formed through its outer wall 23. These may include the intermediate ports 25a, 25b, and 25c, and an end port 25d that may be formed at the distal tip of tube 21. Each port 25a-25d may correspond respectively to one of the lumens 15a-15d. That is, each lumen may define an independent channel extending from one of the infusion ports 11a-11d to one of the tube ports 25a-25d. The sensor assembly may be presented to the sensing environment via positioning at one or more of the ports to provide contact with the medium to be analyzed.

Central line catheters may be known in the art and typically used in the Intensive Care Unit (ICU)/Emergency Room of a hospital to deliver medications through one or more lumens of the catheter to the patient (different lumens for different medications). A central line catheter is typically connected to an infusion device (e.g. infusion pump, IV drip, or syringe port) on one end and the other end inserted in one of the main arteries or veins near the patient's heart to deliver the medications. The infusion device delivers medications, such as, but not limited to, saline, drugs, vitamins, medication, proteins, peptides, insulin, neural transmitters, or the like, as needed to the patient. In alternative embodiments, the central line catheter may be used in any body space or vessel such as intraperitoneal areas, lymph glands, the subcutaneous, the lungs, the digestive tract, or the like and may determine the analyte or therapy in body fluids other than blood. The central line catheter may be a double lumen catheter. In one aspect, an analyte sensor is built into one lumen of a central line catheter and is used for determining characteristic levels in the blood and/or bodily fluids of the user. However, it will be recognized that further embodiments may be used to determine the levels of other agents, characteristics or compositions, such as hormones, cholesterol, medications, concentrations, viral loads (e.g., HIV), or the like. Therefore, although aspects disclosed herein may be primarily described in the context of glucose sensors used in the treatment of diabetes/diabetic symptoms, the aspects disclosed may be applicable to a wide variety of patient treatment programs where a physiological characteristic is monitored in an ICU, including but not limited to blood gases, pH, temperature and other analytes of interest in the vascular system.

In another aspect, a method of intravenously measuring an analyte in a subject is provided. The method comprises providing a catheter comprising the sensor assembly as described herein and introducing the catheter into the vascular system of a subject. The method further comprises measuring an analyte.

Referring now to FIGS. 6-7, sensor embodiments comprising optical fibers are depicted. In FIG. 6, optical fibers 60a, 60b transmit light from light sources 61a, 61b to a bodily fluid sample 25c′ positioned in the port 25c. In some embodiments, the light sources 61a, 61b transmit light at one or more wavelengths. For example, the light source 61a may transmit light at a wavelength of 805 nm and the light source 61b may transmit light at a wavelength of 905 nm Although two light sources and two optical fibers for transmitting light from the light sources are illustrated, the sensor can include any number of light sources and optical fibers. For example, the sensor may include one or more light source and one or more optical fibers. The light sources 61a, 61b may be LEDs, lasers, or any other source capable of generating light over a range of wavelengths and may also include an optical filter for preventing light of undesirable wavelengths from reaching bodily fluid sample 25c′. Optical fiber 60c transmits the light from the bodily fluid sample 25c′ to a photocell 62 for measurement of the light transmitted, absorbed, or scattered through sample 25c′. Photocell 62 sends an electrical signal to the ADC 32 to digitize the signals prior to input to the processor 34. Algorithms programmed in the processor 34 can determine the level of hematocrit in the sample 25c′ based on the signal. The signal may also be used, for example, to calculate a correction factor for adjusting a measured analyte concentration value

FIG. 7 depicts a cross-sectional side view of an alternative sensor embodiment adapted to a central line catheter comprising a sensor or sensor assembly and optical fibers. The optical fibers 60a, 60b, 60c are positioned in lumen 66a of a catheter. Although a multi-lumen catheter is depicted, a single lumen catheter may be used. The optical fibers 60a, 60b are positioned at one side 68a of the port 25c to transmit light to the sample 25c′ in the port 25c. Positioned on opposing side 68b of the port 25c is reflective surface 64 (e.g., a mirror). The reflective surface 64 transmits the light passing through the sample 25c′ to the optical fiber 60c, which is positioned on the one side 68a of the port 25c to receive the light passing through the sample 25c′ and transmit the light to the photocell 62. Although a reflective surface for transmitting the light passing through the sample is illustrated, the light passing through the sample may also be transmitted, for example, by positioning the optical fiber 60c on the opposing side 68b of the port 25c. In the illustrated embodiment, sensor 9 and the wires 47 are positioned in port 25a. In the illustrated embodiment, the sensor 9 comprises at least one electrode. The sensor 9 may comprise a flex circuit or a wire electrode assembly. In other embodiments, the optical fibers 60a, 60b, 60c and/or sensor 9 are positioned at the end port 25d.

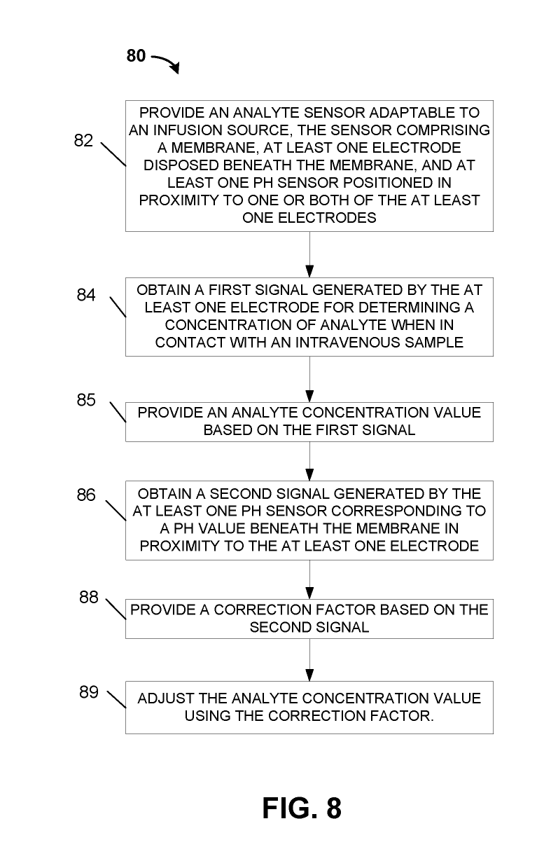

FIG. 8 illustrates a method 80 of adjusting an analyte concentration according to an embodiment. In block 82, a sensor adaptable to an infusion source is provided. The sensor, as disclosed herein, comprises a membrane, at least one electrode disposed beneath the membrane, and at least one pH sensor disposed beneath the membrane and in proximity to the at least one electrode. The membrane includes the active enzymatic portion 50 and inactive-enzymatic or non-enzymatic portion 52 as described hereinabove. In one embodiment, the at least one electrode is disposed beneath one or both of the active enzymatic portion 50 and the inactive-enzymatic or non-enzymatic portion 52 and the at least one pH sensor is disposed beneath the membrane in proximity to the at least one electrode. For example, the pH sensor may be positioned in close proximity to a working electrode under the active enzymatic portion 50.

In block 84, a first signal generated by the at least one electrode is obtained. The first signal is used for determining a concentration of analyte when the at least one electrode is in contact with an intravenous sample. For example, the working electrode 12 can be used to provide a signal that corresponds with the amount of glucose in the bodily fluids of a subject. In block 85, an analyte concentration value based on the first signal is provided. For example, the oxidation of hydrogen peroxide produced as a result of a glucose oxidase reaction at the working electrode results in an electrical current produced as a signal that can be used to calculate a glucose concentration value.

In block 86, a second signal generated by the at least one pH sensor corresponding to a pH value beneath the membrane and in proximity to the at least one electrode is obtained. For example, the concentration of hydrogen ions in the intravenous sample or the infusion source corresponds to a signal produced by the pH sensor corresponding to a pH value. In block 88, a correction factor based on the second signal is provided. In some embodiments, the correction factor is determined by a pH correction curve programmed into an algorithm.

In block 89, the analyte concentration value is adjusted using the correction factor. The correction factor takes into account any variance in enzymatic activity that results from the pH of the intravenous sample and/or infusion source. In addition, other parameters affecting the measurement of the analyte, such as hematocrit as discussed in regard to FIG. 3D, can also be used in the adjustment of the analyte concentration value. In some embodiments, a signal corresponding to a hematocrit level present in bodily fluids is obtained and the measured analyte concentration value is adjusted based on the determined hematocrit level. The impedance value of the bodily fluid corresponding to hematocrit levels is measured and the measured impedance value is used to calculate a second correction factor. The second correction factor is be used to adjust the measured analyte concentration value accordingly.

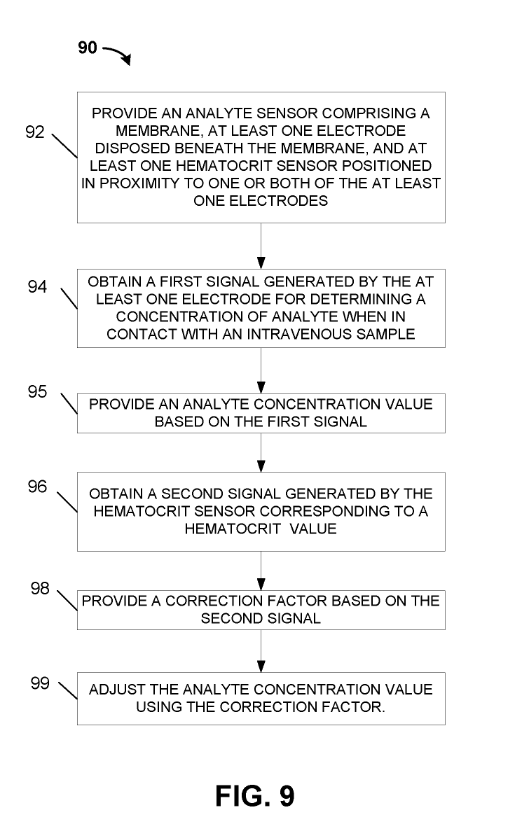

FIG. 9 illustrates a method 90 of adjusting an analyte concentration according to an embodiment. In block 92, a sensor is provided. The sensor, as disclosed herein, comprises a membrane, at least one electrode disposed beneath the membrane, and at least one hematocrit sensor positioned in proximity to the at least one electrode. The membrane includes the active enzymatic portion 50 and inactive-enzymatic or non-enzymatic portion 52 as described hereinabove. In one embodiment, the at least one electrode is disposed beneath one or both of the active enzymatic portion 50 and the inactive-enzymatic or non-enzymatic portion 52 and the hematocrit sensor is disposed beneath the membrane in proximity to the at least one electrode. In other embodiments, the hematocrit sensor comprises four electrodes positioned in proximity to the at least one electrode disposed beneath the membrane. In other embodiments, the at least one electrodes is disposed on one side of a substrate and the hematocrit sensor is disposed on the opposing side of the substrate. In another embodiment, the hematocrit sensor comprises at least one optical fiber.

In block 94, a first signal generated by the at least one electrode is obtained. The first signal is used for determining a concentration of analyte when the at least one electrode is in contact with an intravenous sample. For example, the working electrode 12 can be used to provide a signal that corresponds with the amount of glucose in the bodily fluids of a subject. In block 95, an analyte concentration value based on the first signal is provided. For example, the oxidation of hydrogen peroxide produced as a result of a glucose oxidase reaction at the working electrode results in an electrical current produced as a signal that can be used to calculate a glucose concentration value.

In block 96, a second signal generated by the hematocrit sensor corresponding to a hematocrit level value is obtained. For example, an impedance value or a transmittance value of light passing through a sample is measured to determine a level of hematocrit. In block 98, a correction factor based on the second signal is provided. The correction factor may be determined using an algorithm. And in block 99, the analyte concentration value is adjusted using the correction factor.

Accordingly, sensors and methods have been provided for measuring an analyte in a subject, including a sensor assembly configured for adaption to a continuous glucose monitoring device or a catheter for insertion into a subject's vascular system having electronics unit electrically configurable to the sensor assembly.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

All numbers expressing quantities of ingredients, reaction conditions, and so forth used in the specification may be to be understood as being modified in all instances by the term “about.” Accordingly, unless indicated to the contrary, the numerical parameters set forth herein may be approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials. These descriptions are susceptible to modifications in the methods and materials, as well as alterations in the fabrication methods and equipment. Such modifications will become apparent to those skilled in the art from a consideration of this disclosure or practice of the disclosure. Consequently, it is not intended that this disclosure be limited to the specific embodiments disclosed herein, but that it cover all modifications and alternatives coming within the true scope and spirit of the claims.

What is claimed is:

1. An analyte sensor comprising:
a membrane comprising an active enzymatic portion and an inactive-enzymatic or non-enzymatic portion;
at least two electrodes disposed beneath the membrane; and
at least one pH sensor disposed beneath the membrane and in proximity to the at least two electrodes.

2. The sensor of claim 1, wherein the at least one pH sensor is disposed beneath the membrane.

3. The sensor of any one of the previous claims, wherein the at least two electrodes comprises a working electrode and a blank electrode, and the membrane is partitioned over the working electrode and the blank electrode.

4. The sensor of claim 2, wherein the working electrode is disposed under the active enzymatic portion of the membrane and the blank electrode is disposed under the inactive-enzymatic or non-enzymatic portion of the membrane.

5. The sensor of claim 2, wherein the membrane is partitioned over the working electrode associated with the active enzymatic portion and the blank electrode associated with the inactive-enzymatic or non-enzymatic portion.

6. The sensor of claim 2, wherein the at least one pH sensor is positioned in closer proximity to the working electrode than the blank electrode.

7. The sensor of claim 2, wherein at least one pH sensor is positioned in closer proximity to the blank electrode than the working electrode.

8. The sensor of claim 2, wherein the at least one pH sensor is positioned at an equal distance from the working electrode and the blank electrode.

9. The sensor of claim 1, wherein the active enzymatic portion of the membrane comprises glucose oxidase.

10. The sensor of claim 2, wherein the working electrode and the at least one pH sensor is disposed on a first surface of a sensor substrate.

11. The sensor of claim 2, wherein the working electrode is disposed on a first surface of a sensor substrate, and the at least one pH sensor is disposed on a second surface of the sensor substrate.

12. The sensor of claim 1, wherein the membrane further comprises at least one of an electrode layer, an interferent layer, and a flux limiting layer.

13. The sensor of claim 1, wherein the at least one pH sensor is configured to determine a pH value of an environment in proximity to the at least two electrodes beneath the membrane.

14. A method comprising:
providing an analyte sensor adaptable to an infusion source, the sensor comprising:
a membrane layer comprising one or both of an active enzymatic portion and an inactive-enzymatic or non-enzymatic portion;
at least one working electrode disposed beneath one or both of the active enzymatic portion of the membrane and the inactive-enzymatic or non-enzymatic portion of the membrane; and
a pH sensor positioned in proximity to one or both of the at least one working electrode;
obtaining a first signal generated by the at least one electrode for determining a concentration of an analyte when in contact with an intravenous sample and providing an analyte concentration value based on the first signal;
obtaining a second signal generated by the pH sensor corresponding to a pH value beneath the membrane in proximity to the at least one working electrode;
providing a correction factor based on the second signal; and
adjusting the analyte concentration value using the correction factor.

15. The method of claim 14, wherein the analyte sensor is an intravenous blood glucose sensor (IVBG).

16. The method of claim 14, wherein the correction factor is determined using an algorithm.

17. The method of claim 14, wherein the algorithm comprises a pH correction curve.

18. The method any one of claims 14-17, wherein the second signal corresponds to one or more of the pH of the infusion source introduced to the analyte sensor or the pH of the intravenous sample.

19. The method of claim 18, wherein the pH of the infusion source differs from the pH of the intravenous sample.

20. The method any one of claims 14-17, further comprising obtaining a signal corresponding to a hematocrit level present in the bodily fluid and adjusting the calculated analyte concentration value based on the determined hematocrit level.

21. The method of claim 20 further comprising the steps of:
measuring an impedance value of the bodily fluid corresponding to a hematocrit level;
calculating a second correction factor based on the measured impedance value; and
adjusting the calculated analyte concentration value based on the calculated second correction factor.

22. The method of claim 21, wherein the calculated analyte concentration value is adjusted based on the calculated first correction factor and the calculated second correction factor.

23. The method of claim 14, wherein the pH sensor is disposed beneath the membrane.

24. The method of claim 14, wherein the pH sensor is disposed beneath an ion-sensitive membrane.

25. A system comprising:
an intravenous analyte sensor adapted for fluid communication with an infusion fluid source and intravenous fluids, the analyte sensor comprising:
at least one enzyme electrode configured to generate a first signal, corresponding to an analyte concentration value of the intravenous fluid; and
at least one pH sensor in proximity to the at least one enzyme electrode, the pH sensor configured to generate a second signal corresponding to a pH value of one or more of the infusion fluid source and the intravenous fluid; and
wherein the system is configured to adjust the analyte concentration value based on the pH value corresponding to the second signal.
